(19) United States
(12) Patent Application Publication
(10) Pub. No.: US 2016/0331304 A1
(43) Pub. Date: Nov. 17, 2016

(54) SYSTEM AND METHODS FOR AUTOMATED ADMINISTRATION AND EVALUATION OF PHYSICAL THERAPY EXERCISES

(71) Applicant: University of Washington, Seattle, WA (US)

(72) Inventors: Tyler Libey, Seattle, WA (US); Lars Crawford, Seattle, WA (US); Dimitrios C. Gklezakos, Seattle, WA (US); Brian Mogen, Seattle, WA (US)

(73) Assignee: University of Washington, Seattle, WA (US)

(21) Appl. No.: 15/154,382

(22) Filed: May 13, 2016

Related U.S. Application Data
(60) Provisional application No. 62/161,100, filed on May 13, 2015.

(51) Int. Cl. A61B 5/00; A61B 5/11; A61B 5/0488

(52) U.S. Cl. CPC A61B 5/486; A61B 5/0488; A61B 5/742; A61B 5/1114; A61B 5/4848; A61B 2560/0223; A61B 2505/09

(57) ABSTRACT

Methods and systems for automated administration and evaluation of physical therapy exercises are provided. A motion prompt is displayed on a display device. Hand position information is generated by a hand position sensing device, and muscular activity information is generated by a muscular activity sensing device, while a user performs the prompted motion. Compliance with the motion prompt is determined by analyzing the hand position information, and muscular activity is evaluated using the muscular activity information. The difficulty of the motion prompts provided by the system is automatically configurable based on the determined compliance with the motion prompt and the muscular activity evaluation.

### CROSS-REFERENCE TO RELATED APPLICATION

This application claims the benefit of Provisional Application No. 62/161,100, filed May 13, 2015, the entire disclosure of which is hereby incorporated by reference herein for all purposes.

### BACKGROUND

A large number of patients are currently in hand rehabilitation therapy in the United States as the result of stroke, and this number is projected to grow due to the aging demographic. In 2010, stroke related medical and disability costs totaled $73.3 billion in the United States. Nearly $8 billion of which is spent on hand related rehabilitation alone, as almost 67% of strokes cause forearm motor losses. When a stroke affects a patient's hand function, recovery is slow and gains are incremental and difficult to measure. Patients typically progress through inpatient to outpatient and at-home therapy over the course of six to ten weeks. The slow, immeasurable progress can lead to a premature loss of insurance reimbursement for therapy procedures and low patient motivation and compliance. The almost 17,000 rehab clinics in the United States struggle to provide adequate support for these patients because of the difficulty in tracking improvements and covering the cost of continued outpatient therapy. What are needed are systems and methods that automate the administration of physical and/or occupational therapy procedures, post-surgery assessments, post-operative care, and/or the like (herein collectively referred to as “physical therapy” for ease of discussion) in order to improve compliance rates and reduce the cost of administration. It is also desirable to create systems that can evaluate the performance of physical therapy tasks in a more precise way than is currently available in order to be able to measure and demonstrate small amounts of incremental progress.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a system for automated administration and evaluation of physical therapy exercises is provided. The system comprises a computing device, a display device, a hand position sensing device, and a muscular activity sensing device. The computing device comprises at least one processor and a non-transitory computer readable medium. The display device is communicatively coupled to the computing device. The hand position sensing device is communicatively coupled to the computing device. The muscular activity sensing device is communicatively coupled to the computing device and configured to sense muscular activity in a forearm of a user.

In some embodiments, a computer-implemented method for automated administration and evaluation of physical therapy exercises is provided. A computing device presents, via a display device, a motion prompt. The computing device receives hand position information from a hand position sensing device. The computing device receives muscular activity information from a muscular activity sensing device. The computing device determines compliance with the motion prompt using the hand position information. The computing device determines a muscular activity evaluation score based on the muscular activity information.

In some embodiments, a nontransitory computer-readable medium having computer-executable instructions stored thereon is provided. The instructions, in response to execution by one or more processors of a computing device, cause the computing device to perform actions comprising: presenting, by the computing device via a display device, a motion prompt; receiving, by the computing device, hand position information from a hand position sensing device; receiving, by the computing device, muscular activity information from a muscular activity sensing device; determining, by the computing device, compliance with the motion prompt using the hand position information; and determining, by the computing device, a muscular activity evaluation score based on the muscular activity information.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram that illustrates an exemplary embodiment of a system for automated administration and evaluation of physical therapy exercises according to various aspects of the present disclosure;

FIG. 2 is a block diagram that illustrates details of devices within an exemplary embodiment of a system according to various aspects of the present disclosure;

FIG. 3A illustrates an example Ball Roll motion prompt and an associated hand position according to various aspects of the present disclosure;

FIG. 3B illustrates an example of a Five Finger Mole motion prompt and an associated hand position according to various aspects of the present disclosure;

FIG. 4 is a perspective view of an exemplary embodiment of a muscular activity sensing device suitable for use with the system for automated administration and evaluation of physical therapy exercises;

FIGS. 5A and 5B are a flowchart that illustrates an exemplary method of automated administration and evaluation of physical therapy exercises according to various aspects of the present disclosure;

FIG. 6 is a flowchart that illustrates an exemplary embodiment of a method for calibrating data generated by a muscular activity sensing device according to various aspects of the present disclosure; and

FIG. 7 is a block diagram that illustrates aspects of an exemplary computing device 700 appropriate for use with embodiments of the present disclosure.

### DETAILED DESCRIPTION

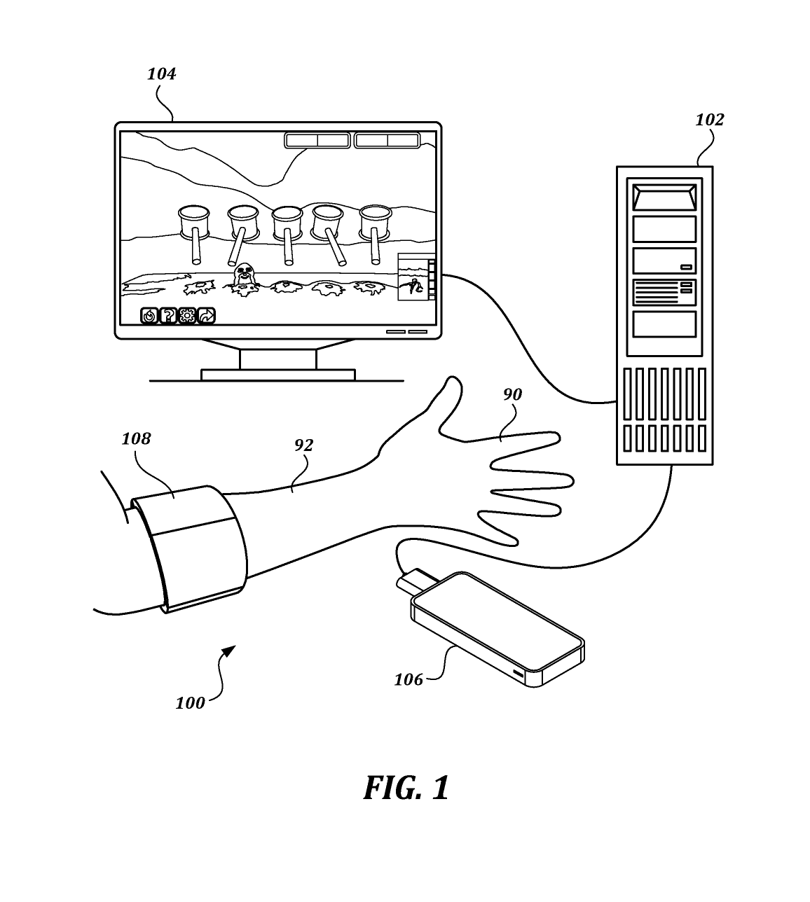

FIG. 1 is a schematic diagram that illustrates an exemplary embodiment of a system for automated administration and evaluation of physical therapy exercises according to various aspects of the present disclosure. The system 100 includes a computing device 102, a display device 104, a hand position sensing device 106, and a muscular activity sensing device 108. A user places the muscular activity sensing device 108 on their arm 92, and moves their hand 90 to be positioned within the sensing range of the hand position sensing device 106. The computing device 102 causes motion prompts to be displayed on the display device Q04, and the user moves their hand 90 and arm 92 as prompted. The hand position sensing device 106 generates data regarding the position of the hand 90 and arm 92 in three-dimensional space, and provides the data to the computing device 102. The muscular activity sensing device 108 generates data regarding muscular activity within the arm 92, and provides the data to the computing device 102 as well. The system 100 can thereby guide the user through one or more physical therapy exercises, ensure that the user has performed the exercises as prompted, and evaluate muscular performance and coordination while the exercises are performed. Though a single arm 92 is illustrated, in some embodiments both arms of the user may be used and tracked by the system 100.

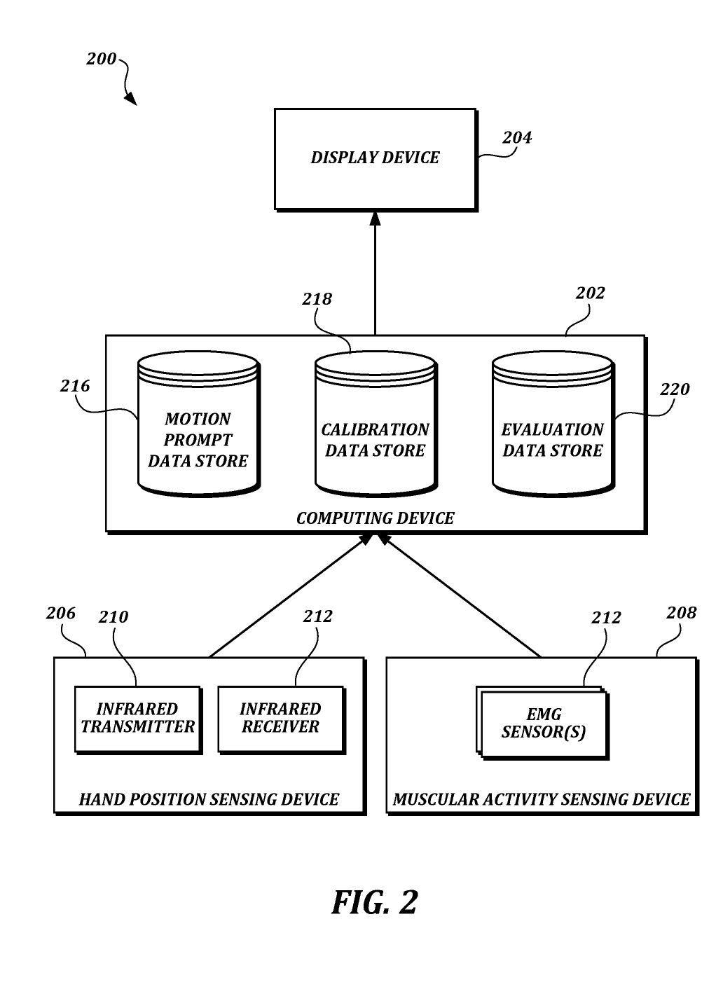

FIG. 2 is a block diagram that illustrates details of devices within an exemplary embodiment of a system according to various aspects of the present disclosure. As illustrated, the system 200 includes a display device 204, a computing device 202, a hand position sensing device 206, and a muscular activity sensing device 208.

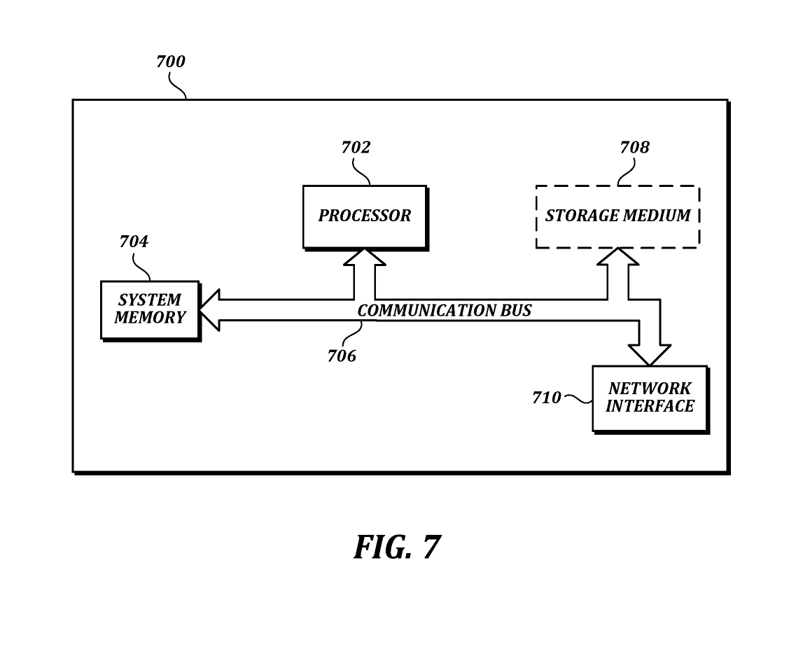

The computing device 202 may be any suitable type of computing device, including but not limited to a desktop computing device, a laptop computing device, a tablet computing device, a smartphone computing device, and/or any other suitable type of computing device for storing, receiving, and processing data as described below. Further components common to all computing devices are described below with respect to FIG. 7. In addition to these components, the computing device 202 is configured to provide (or provide access to) a motion prompt data store 216, a calibration data store 218, and an evaluation data store 220.

The motion prompt data store 216 is configured to store motion prompt information for a plurality of motion prompts, wherein the motion prompt information defines various factors of how a motion prompt should be presented and how compliance with the motion prompt should be evaluated. Typically, the motion prompts include animated, interactive games that encourage particular types of physical hand motion. Some examples of motion prompts are a Five Finger Mole prompt, a Ball Roll prompt, a Reach and Grab prompt, a Hand Match prompt, and a vAssess prompt.

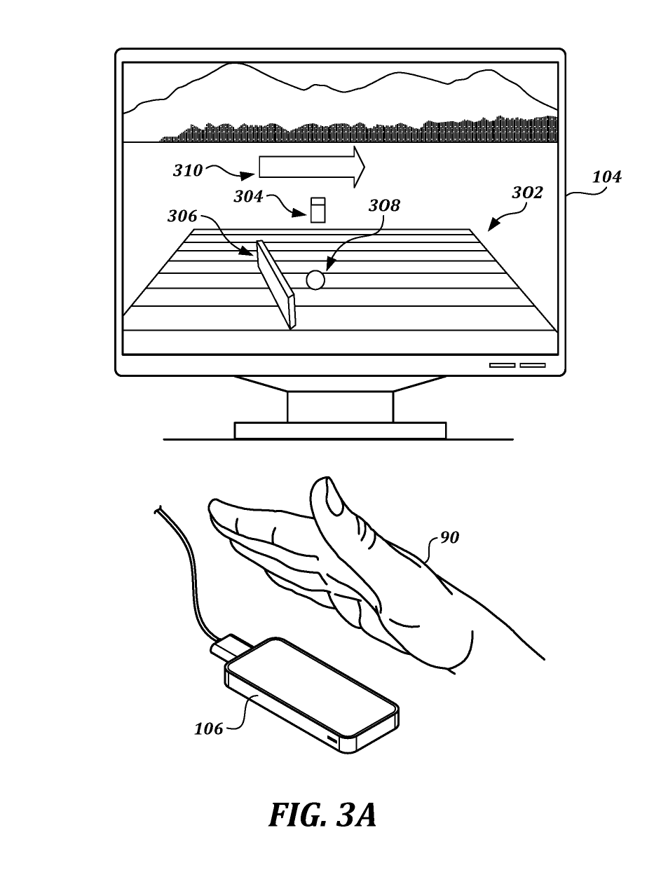

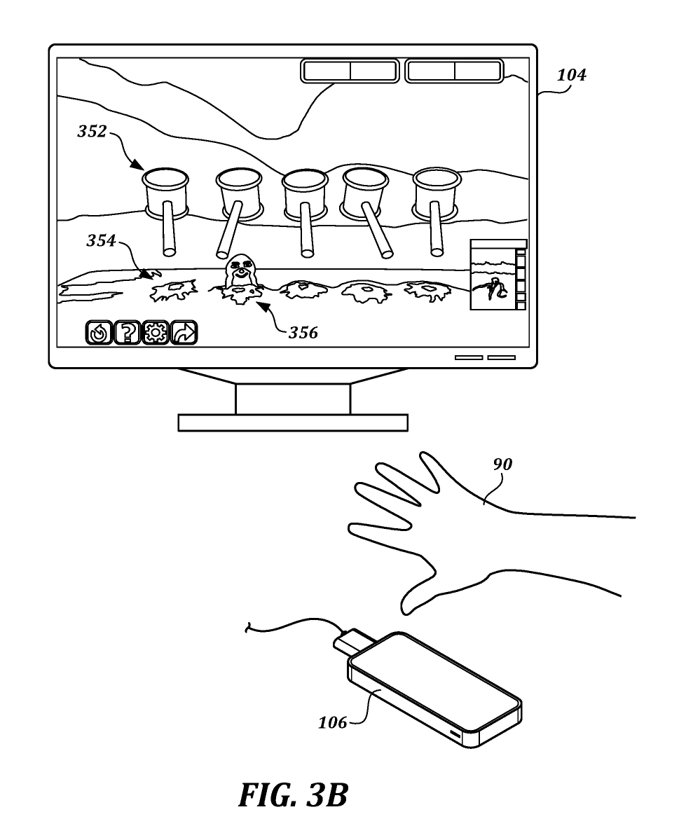

FIG. 3A illustrates an example Ball Roll motion prompt and an associated hand position according to various aspects of the present disclosure. The Ball Roll prompt displays a table 302 with a vertical paddle 306 at or near the middle. The user is prompted to hold their hand 90 vertically, and to flex/extend their wrist to rotate the paddle 306. As a ball 308 rolls down the table 302 from a launcher 304, rotating the paddle 306 by flexing/extending the user's wrist causes the ball 308 to be hit to one side of the table or the other as instructed by an arrow 310. This activity can test flexion and extension of the wrist, among other things.

FIG. 3B illustrates an example of a Five Finger Mole motion prompt and an associated hand position according to various aspects of the present disclosure. The Five Finger Mole prompt displays a game field reminiscent of the game “whack-a-mole,” having a set of holes 354 from which animated moles 356 may pop up. A set of hammers 352 corresponding to each of the user's fingers is positioned above each of the holes 354. When a mole 356, the user moves the associated finger (for example, in the illustrated case, the index finger) vertically in order to move the associated hammer to hit the mole 356. This activity can test range of motion, coordination, and reaction time for each individual finger of the hand 90.

Other prompts are also available: The Reach and Grab prompt displays a grid of cubby holes with balls inside. A virtual hand that mimics the location and orientation of the user's hand is also displayed. The user is prompted to reach out and form fists in order to cause the virtual hand to grab the balls out of the cubby holes. The Hand Match prompt displays a similar virtual hand that mimics the location and orientation of the user's hand, and also displays a prompt hand that changes position and orientation. The user is prompted to move their hand to match the position and orientation of the prompt hand. The Hand Match prompt may test both hands at the same time. The vAssess prompt illustrates the virtual hand, as well as a set of bubbles to be popped by touching them with the virtual hand.

The calibration data store 218 is configured to store canonical data sets and calibration data that allow data collected by the muscular activity sensing device 208 from multiple different sessions to be compared to each other, regardless of how the muscular activity sensing device 208 is positioned on the user's arm. Further discussion of the creation and use of canonical data sets and calibration data is provided below.

The evaluation data store 220 is configured to store evaluation scores that represent evaluations of how successfully the user was able to comply with motion prompts. The evaluation scores may then be used to determine rehabilitation progress, to tune the system 200 for future sessions, and/or for any other use. Further discussion of the evaluation scores is provided below.

As understood by one of ordinary skill in the art, a “data store” as described herein may be any suitable device configured to store data for access by a computing device. One example of a data store suitable for use with the computing device 202 is a highly reliable, high-speed relational database management system (RDBMS) executing on one or more computing devices and accessible over a high-speed network. However, any other suitable storage technique and/or device capable of quickly and reliably providing the stored data in response to queries may be used, such as a key-value store, an object database, and/or the like. Further, the computing device providing the data store may be accessible locally by the computing device 202 instead of over a network, or may be provided by the computing device 202 itself. A data store may also include data stored in an organized manner on a computer-readable storage medium, as described further below. One example of such a data store is a file system or database management system that stores data in files (or records) on a computer readable medium such as flash memory, random access memory (RAM), hard disk drives, and/or the like. One of ordinary skill in the art will recognize that separate data stores described herein may be combined into a single data store, and/or a single data store described herein may be separated into multiple data stores, without departing from the scope of the present disclosure.

The display device 204 may be any type of display device capable of presenting video and/or audio motion prompts. Typically, the display device 204 is a monitor connected to the computing device via HDMI, FireWire, DisplayPort, VGA, USB, or any other suitable communication technology. In some embodiments, the display device 204 may be incorporated into the computing device 202, such as in embodiments wherein the computing device 202 is a laptop computing device, a tablet computing device, or a smartphone computing device.

The figures and description below primarily relate to such embodiments wherein the display device 204 is a monitor. However, in some embodiments, instead of being a monitor the display device 204 may be a virtual reality headset, such as the Rift headset created by Oculus VR, the Vive headset created by HTC Corporation, The PlayStation VR headset by Sony Corporation, the Gear VR headset by Samsung, and/or the like. In some embodiments, the display device 204 may be an augmented reality headset such as the Glass headset created by Google Inc., the HoloLens headset created by Microsoft Corporation, and/or the like. Any technology capable of presenting a motion prompt may be used for the display device 204.

The hand position sensing device 206 is any device capable of generating three-dimensional position information that represents the elements of a user's hand or hands. In the illustrated embodiment, the hand position sensing device 206 includes at least one infrared transmitter 210 and at least one infrared receiver 212. In some embodiments, at least two infrared receivers 212 may be used in order to obtain stereo images and thereby collect three-dimensional position information. One example of a device suitable for use as a hand position sensing device 206 within the system 200 is a Leap Motion controller created by Leap Motion, Inc, though any other suitable device with similar functionality may be used. The hand position sensing device 206 may be communicatively coupled to the computing device 202 using any suitable technique, including but not limited to a USB connection, a FireWire connection, Bluetooth, Bluetooth Low Energy, ZigBee, WiFi, a LAN connection, a WAN connection, and an internet connection.

The muscular activity sensing device 208 is any device capable of generating data indicating muscular activity within the arm of the user. In the illustrated embodiment, the muscular activity sensing device 208 includes a plurality of surface electromyography (EMG) sensors 214 that are capable of determining muscular activity from the surface above the muscle on the skin. In some embodiments, each of the EMG sensors 214 may include two or more electrodes that are used to measure potential differences between particular locations on the skin. One example of a device suitable for use as a muscular activity sensing device 208 within the system 200 is a Myo armband controller created by Thalmic Labs Inc., though any other suitable device with similar functionality may be used. The muscular activity sensing device 208 may be communicatively coupled to the computing device 202 using any suitable technique, including but not limited to a USB connection, a FireWire connection, Bluetooth, Bluetooth Low Energy, ZigBee, WiFi, a LAN connection, a WAN connection, and an internet connection

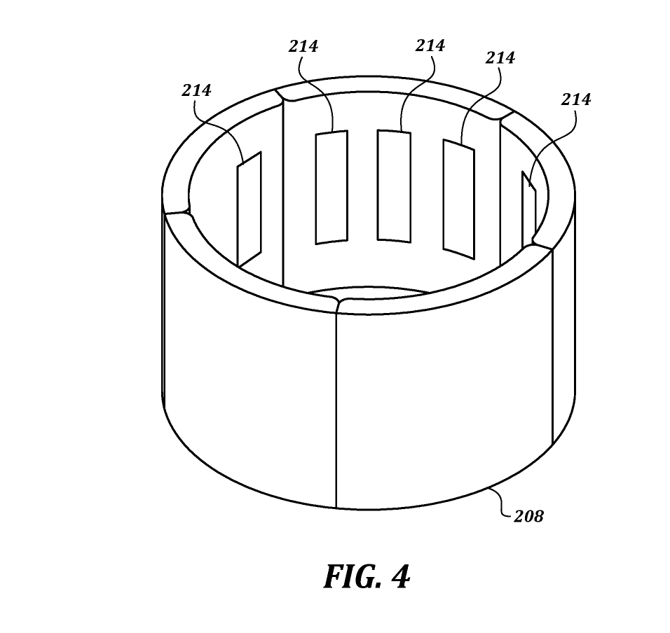

FIG. 4 is a perspective view of an exemplary embodiment of a muscular activity sensing device suitable for use with the system for automated administration and evaluation of physical therapy exercises. The muscular activity sensing device 208 includes a plurality of EMG sensors 214. As is shown, the muscular activity sensing device 208 exhibits rotational symmetry about the lumen through which the arm is inserted. Because the muscular activity sensing device 208 is a bracelet, there is a distinct possibility that the EMG sensors 214 will be placed in a different rotational position on the arm for each session. Embodiments of the present disclosure compensate for different positions as described below, and may use the rotational symmetry of the muscular activity sensing device 208 to enhance the accuracy of the compensation.

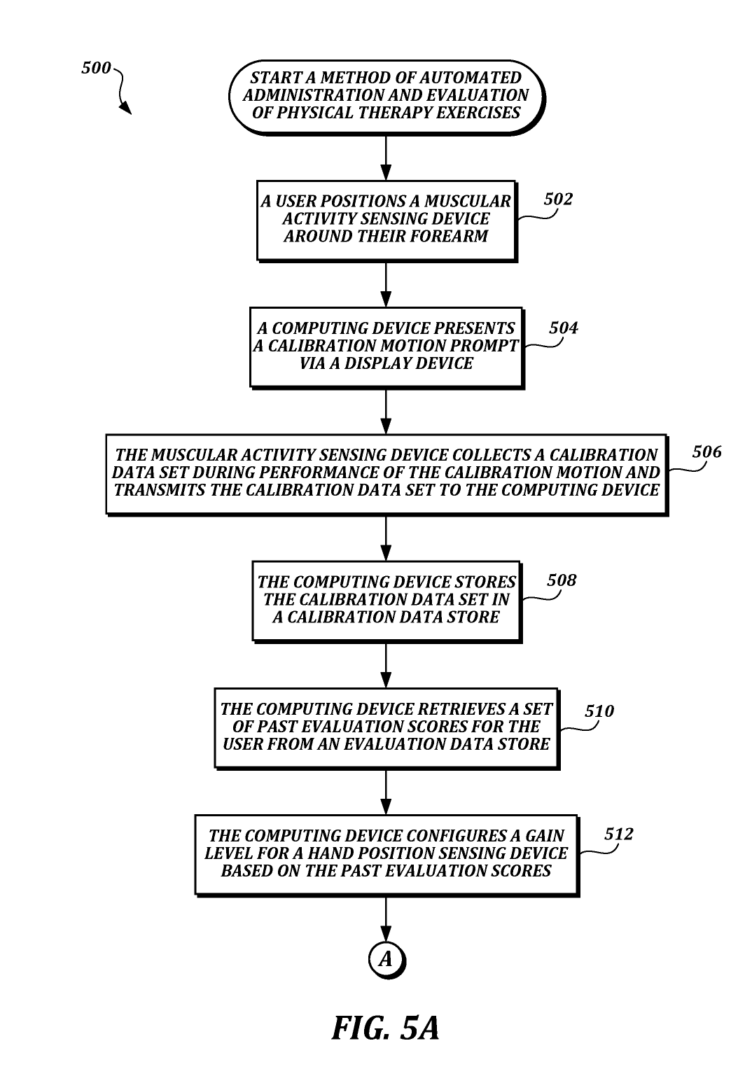

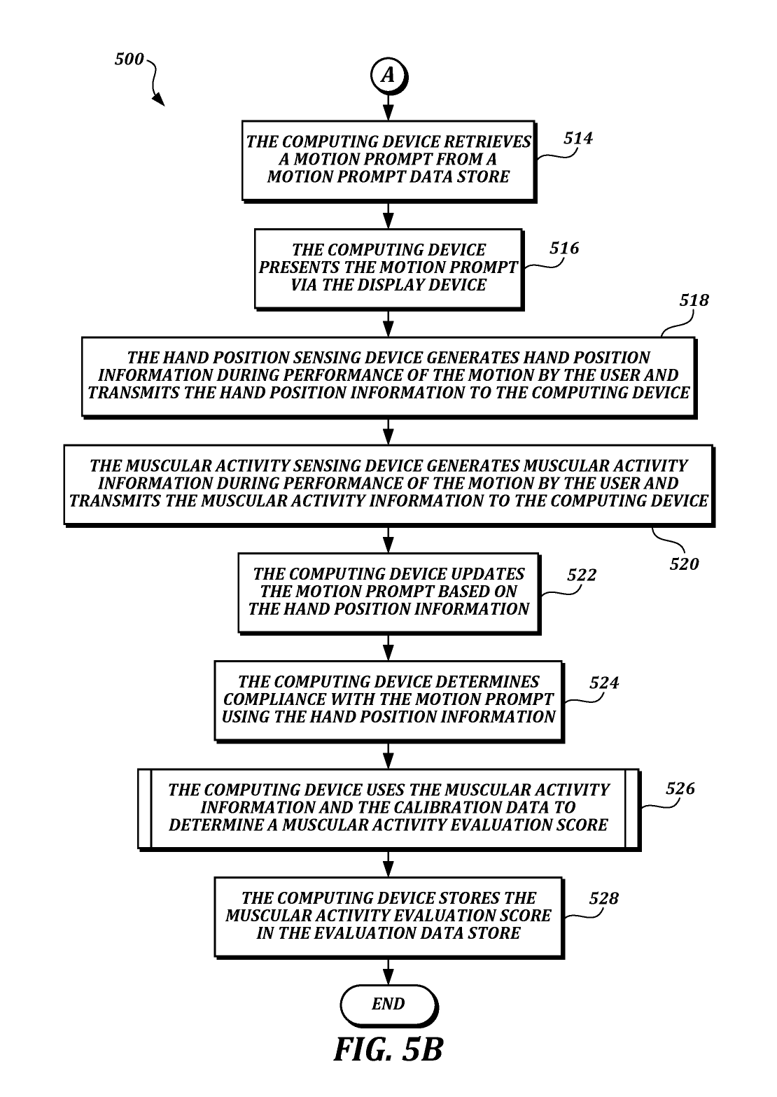

FIGS. 5A and 5B are a flowchart that that illustrates an exemplary method of automated administration and evaluation of physical therapy exercises according to various aspects of the present disclosure. From a start block, the method 500 proceeds to block 502, where a user positions a muscular activity sensing device 208 around their forearm. As discussed above, the rotational position of the muscular activity sensing device 208 may not matter, but instead will be compensated for during the calibration process discussed below. Next, at block 504, a computing device 204 presents a calibration motion prompt via a display device 204. One example of a calibration motion prompt includes animations that prompt the user to perform a sequence such as (1) horizontal flexion; (2) horizontal extension; (3) radial deviation; (4) ulnar deviation; (5) vertical flexion; and (6) vertical extension. Each motion may be prompted to be held for three seconds, and may be followed by a three second rest. In some embodiments, the calibration motion prompt may direct these motions by presenting textual descriptions of the motions. In some embodiments, the calibration motion prompt may direct these motions by presenting animations that depict each desired motion. The calibration movements allow the muscular activity sensing device 208 to gather labeled muscular activity data for each of the movements.

At block 506, the muscular activity sensing device 208 collects a calibration data set during performance of the calibration motion and transmits the calibration data set to the computing device 202. In some embodiments, the hand position sensing device 206 may also transmit hand position information to the computing device 202. At block 508, the computing device 202 stores the calibration data set in a calibration data store 218. In some embodiments, the hand position information may be stored in the calibration data store 218, as well, while in other embodiments, the hand position information may simply be analyzed by the computing device 202 to determine whether the calibration data set should be considered valid. The calibration data set may be used for analyzing data gathered during later exercises, as discussed further below.

The method 500 then proceeds to block 510, where the computing device 202 retrieves a set of past evaluation scores for the user from an evaluation data store 220. In some embodiments, the past evaluation scores may directly indicate difficulty levels that have been successfully completed or gain levels that have been used by the user for one or more motion prompts. In some embodiments, the past evaluation scores may indicate levels of physical ability, such as range of motion, speed of motion, muscular strength or endurance, and/or the like. At block 512, the computing device 202 configures a gain level for a hand position sensing device 206 based on the past evaluation scores. If the past evaluation scores indicate that range of motion is small or the muscular strength is weak, the gain may be set high in order to translate small movements or efforts into large movements on the display. This allows for a feeling of accomplishment to the user, and allows the user to see a demonstration of progress. If the past evaluation scores indicate that the range of motion are large or normal or that the muscular strength is high, the gain may be set low in order to increase the challenge for the user. The past evaluation scores may also be used for other tuning of the system 200, such as selecting types of motion prompts to be presented, selecting a difficulty of motions to be performed, and/or the like. The method 500 then proceeds to a continuation terminal (“terminal A”).

From terminal A (FIG. 5B), the method 500 proceeds to block 514, where the computing device 202 retrieves a motion prompt from a motion prompt data store 216. As stated above, the motion prompt may be selected from a plurality of motion prompts based on past evaluation scores. At block 516, the computing device 202 presents the motion prompt via the display device 204. At block 518, the hand position sensing device 206 generates hand position information during performance of the motion by the user and transmits the hand position information to the computing device 202.

In some embodiments, the hand position information includes three-dimensional location information and orientation information for one or more anatomical elements of a hand. For example, objects representing the three-dimensional location one or more hand bones (such as distal phalanges, intermediate phalanges, proximal phalanges, metacarpals, and so on) may be included in the hand position information. As another example, other information relating to the overall position of the hand, such as a vector orthogonal to the face of the palm, a vector indicating arm position, a vector indicating elbow position, a vector indicating wrist position, and so on, may be provided.

At block 520, the muscular activity sensing device 208 generates muscular activity information during performance of the motion by the user and transmits the muscular activity information to the computing device 202. In some embodiments, the muscular activity information may be provided by the muscular activity sensing device 208 in a raw form as a stream of electrical potential values obtained by each of the EMG sensors 214.

At block 522, the computing device 202 updates the motion prompt based on the hand position information. For example, the computing device 202 may cause an on-screen item, such as a paddle, a hammer, or a representation of a hand, to move to mimic the motion represented by the hand position information. The computing device 202 may also alter other objects within the prompt, such as balls, targets, and/or the like, based on the motion represented by the hand position information. At block 524, the computing device 202 determines compliance with the motion prompt using the hand position information. For example, the computing device 202 may perform collision detection between objects within the motion prompt to determine if a goal (such as grabbing a ball with a virtual hand, hitting a virtual target, and/or the like) was achieved. As another example, the computing device 202 may perform a measurement of hand/arm position compared to a standard, such as a flexion of at least 30 degrees, and an extension of at least 30 degrees,” regardless of the presentation of the motion prompt.

In some embodiments, particular metrics are computed for each of the motion prompts to evaluate performance. For each of the metrics, the metric may be evaluated in comparison to a predetermined threshold that indicates a successful completion of the motion. The computing device 202 may use the determined compliance to determine game actions, such as advancing to a more difficult level in a game, repeating a current level of difficulty, reducing the level of difficulty (or increasing the gain), and/or the like.

For the Five Finger Mole prompt, the system 200 may determine individual finger range of vertical motion, finger independence, average trial for each finger in terms of vertical position, and mean and standard deviation of reaction time. Finger independence may be measure in two ways. The first technique is via a correlation matrix of vertical finger movement. The second technique is via the distance covered by other fingers relative to the finger associated with a given trial. For example, for a thumb trial, the distance travelled by each finger is divided by the distance traveled by the thumb.

For the Ball Roll prompt, the system 200 may determine minimum and maximum angle of the wrist, minimum and maximum wrist angle trimmed by standard deviation, and average flexion and extension in terms of angle. To compute the average flexion and extension in terms of angle, the minimum or maximum wrist angle during a trial is determined and an interval is centered around it. The interval is extended to reach the beginning or the end of the trial window, whichever is closer. If the interval length exceeds a predetermined threshold, the trial is included in the computation of the average trial, otherwise it is rejected. The number of flexions and extensions included for this computation may be saved to get a sense of how accurate it is.

For the Reach and Grab prompt, multiple “intervals” are measured, wherein an interval is what takes place between putting an item in a bin and the last time the user touched the item. These intervals may be pruned by depth using standard deviation to exclude intervals where the item was dropped and picked up at a later time. The end-points of the intervals in virtual 3D space may be clustered via k-means to figure out where the shelves lie in the real volume sensed by the hand position sensing device 206. This defines a range-of-motion (ROM) window. The error, defined as the distance from the cluster centroid to which each reach end-point belongs, may be used to measure reach precision. Path accuracy may be computed as the squared distance of the actual path from the straight line between the interval beginning and end. This estimates the quality of path planning. Accuracy may be binned by shelf end-point target (six target). Path tremor may be computed as above, but using a smoothed version of the path instead of a straight line. This estimates how jittery the movement is. Tremor may be binned as above. Each of these measures may be used to evaluate the movement.

For the Hand Match prompt, if the positions of both hands are measured, then a root-mean-square (RMS) of the left and right hand path tremor may be calculated. A vertical distance between the hands over the whole session and on average over various trials may also be measured. An angle of palm normals during the whole session may also be measured.

For the vAssess prompt, a volume of the convex hull of points corresponding to popped balloons may be calculated, which estimates a physical reach. A trimmed max and min angle for radial/ulnar deviation, vertical flexion/extension, horizontal flexion/extension, and supination may also be determined.

One of ordinary skill in the art will understand that the actions described with respect to blocks 516-524 may be performed concurrently and repeatedly for a period of time.

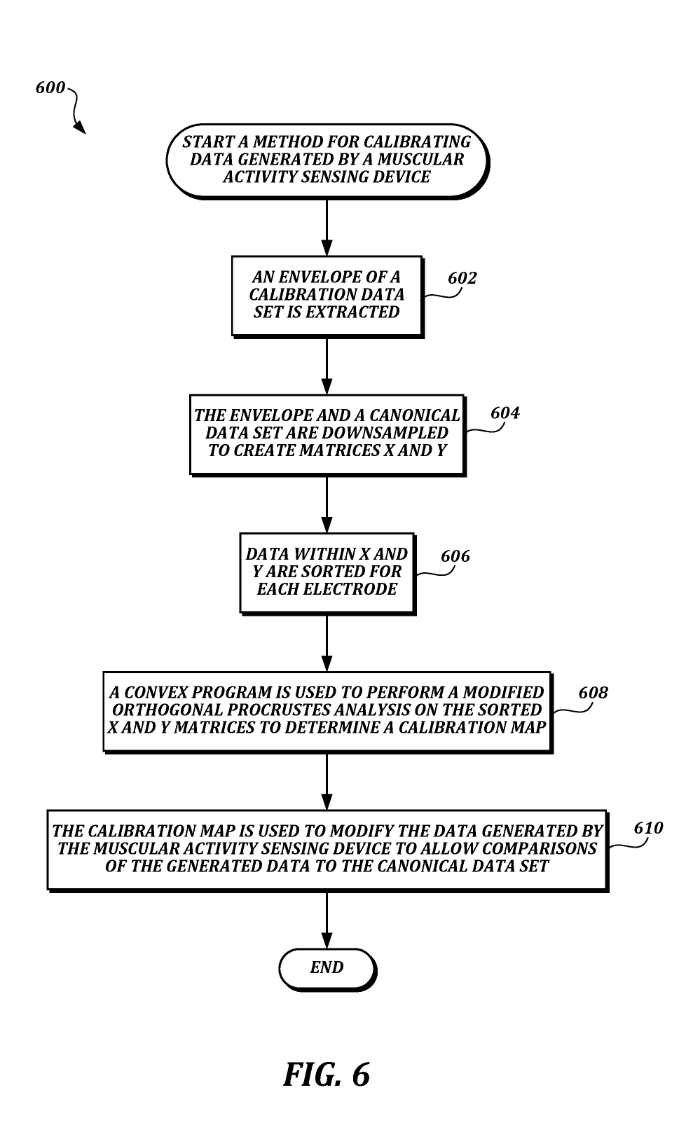

At procedure block 526, the computing device 202 uses the muscular activity information and the calibration data to determine a muscular activity evaluation score. Any suitable technique may be used, and the techniques may be different for different motion prompts. In some embodiments, the muscular activity information may be normalized using the calibration data before using the normalized muscular activity information to determine the muscular activity evaluation score. One example procedure for using the calibration data to normalize the muscular activity data for comparison to previous sessions is illustrated in FIG. 6 and described below.

For determining the muscular activity evaluation score, one example technique relates to the variance explained by muscle synergies. Muscle synergies are a representation of how the nervous system controls complex hand movements, where multiple muscle groups are activated simultaneously to achieve any given action. Instead of individually controlling the various muscles required to perform a movement, the nervous system can generate a movement pattern by combining a (usually small) set of such synergies. In an injury state where the nervous system is damaged and/or reorganizing it is common for the muscle activation patterns to be less complex. Accordingly, a muscular activity evaluation score can be determined by tracking the explained by a reconstructed signal as a function of muscle synergies employed, as a measure of the overall complexity of the muscle activations of the patient. Monitoring the profile of these muscle synergies over time can provide a measure of progress.

With respect to the muscular activity information generated by the system 200, a synergy is represented by an n-dimensional vector that explains the activations of each electrode for each synergy, whereas the synergy activations explain how synergies are employed over time. Specifically, a non-negative matrix factorization of the envelope of the n-dimensional muscular activity information can be performed, wherein n is the number of channels of muscular activity information provided by the muscular activity sensing device 208. One example embodiment of a muscular activity sensing device 208 includes eight EMG sensors 214, and so would produce eight-dimensional information. This models the acquired signal as a collection of constant muscle activation patterns (synergies) scaled by a time-varying activation component. The non-negative matrix factorization decomposes the muscular activity information into n n-dimensional muscle synergies and n-dimensional activations of synergies over time. Some other techniques for determining the muscular activity evaluation score may include cluster distance between clusters of data points for given movements, and the like.

The method 500 then proceeds to block 528, where the computing device 202 stores the muscular activity evaluation score in the evaluation data store 220. In some embodiments, the method 500 may at this point return to block 514 and display another prompt, or display the same prompt with different gain or difficulty settings. Otherwise, the method 500 proceeds to an end block and terminates.

Normalizing the muscular activity data is desirable in order to generate meaningful muscular activity evaluation scores that can be compared to each other from separate sessions, because exact reproduction of EMG electrode placement is inherently hard. This is a significant obstacle for experiments that span sessions between which the user removes the muscular activity sensing device 208. An inter-session analysis of such information will likely result in inexact comparisons due to differences in positioning. As an example, consider the Ball-Roll prompt. During the session, muscular activity information is captured. A subsequent analysis reveals electrode activations during flexion or extension, correlated with the contraction of the muscles directly below or in the surrounding area of each electrode. An example inter-session comparison would be to compare these electrode activations between sessions, as a proxy to contraction strength of the underlying muscles, related to each of the two movement groups, over time. The goal is to enable such an analysis, without requiring precise placement of the bracelet on the forearm. The solution to this problem is to learn a mapping from the electrode positions of one session to the other, effectively translating data between frames of reference.

FIG. 6 is a flowchart that illustrates an exemplary embodiment of a method for calibrating data generated by a muscular activity sensing device according to various aspects of the present disclosure. The method 600 is an example of a method suitable for use in procedure block 526 illustrated in FIG. 5B.

From a start block, the method 600 proceeds to block 602, where an envelope of a calibration data set is extracted. As discussed above with respect to blocks 504-508 in FIG. 5A, the calibration data set may be generated via the performance of a standardized set of movements, and the calibration data set may be stored in the calibration data store 218. The envelope may be extracted using any suitable technique, such as low-pass/high-pass filtering of the signal or a Hilbert transform.

Next, at block 604, the envelope of the calibration data set and an envelope of a canonical data set are downsampled to create matrices X and Y. The canonical data set may be a calibration data set that is stored the first time the system 200 is used by a given user. Alternatively, the canonical data set may be a pre-configured data set that is stored in the system 200 during manufacturing. In some embodiments, the canonical data set may already be in the form of an envelope. In some embodiments, the envelope of the canonical data set may be extracted when needed in a similar manner to the extraction of the envelope of the calibration data set. In some embodiments, the canonical information may be retrieved from the calibration data store 218. The sampling results in two matrices in [symbol] ^{(N}^{₁}^{+ . . . +N}^{_{K}}^{)×d}, where Nᵢ is the number of data points in movement group i and d is the number of electrodes.

Next, at block 606, data within X and Y are sorted for each electrode. This results in matrices X, Yε [symbol] ^{(N}^{₁}^{+ . . . +N}^{_{K}}^{)×d}, with the property that for each movement group i and electrode j, the vector X_{N}_{_{i,j}} is sorted in ascending order. This is done so that each data row of X and Y within the same data group corresponds approximately to the same quantile of the intra-group distribution.

The method 600 then proceeds to block 608, where a convex program is used to perform a modified orthogonal Procrustes analysis on the sorted X and Y matrices to determine a calibration map A. Previous attempts at solving similar problems have used an orthogonal Procrustes approach. However, this approach is not suitable for EMG calibration. A rotation matrix A≠I will surely assign negative coefficients to some of the source electrodes when trying to reconstruct the targets. When the desired analysis depends on non-negative aspects of the signal or the orientation features are non-negative, the translated data obtained from the orthogonal Procrustes will often be negative and therefore unsuitable. Another issue with applying this technique to EMG data is that negative channels do not make sense since we expect the signals to be additive.

A first approach for a convex program suitable for use in determining a calibration map is to modify the orthogonal Procrustes approach by replacing the orthogonality constraint with the positivity constraint A≧0. The convex program formulation of this technique is:

$${min}_{A} {‖ Y - AX ‖}_{F}^{2}$$

such that A≧0. The solution can then be obtained via various constrained convex optimization techniques. One approach that works well for this application is to perform gradient descent on the objective and project the solution at each step to the feasible set by setting all negative elements of A to zero.

Higher quality solutions can be obtained when assumptions can be made regarding the topology of the muscular activity sensing device 208. For example, when the muscular activity sensing device 208 is a bracelet that exhibits rotational symmetry, one can assume that the various electrode positionings are simply rotations of each other. In this context, the quality of the solution can be improved by imposing additional constraints on the solution. Specifically, one can assume that each electrode of the target positioning can be reconstructed by only a few electrodes of the source. This intuition can be added to the model by requiring that each row of A is sparse. One way of incentivizing the model to favor sparse solutions is penalizing the l₁-norm of A given by:

$${‖ A ‖}_{1} = {\sum }_{i , j} \vert {A}_{ij} \vert$$

The resulting convex program is:

$${min}_{A} {‖ Y - AX ‖}_{F}^{2} + λ {‖ A ‖}_{1}$$

such that A≧0, wherein λ is the sparsity hyper-parameter. Larger values of λ favor sparser solutions at the expense of reconstruction quality. A suitable optimization technique for this program is FISTA modified to project the current solution to the feasible set as described above.

At block 610, the calibration map is used to modify the data generated by the muscular activity sensing device 208 to allow comparisons of the generated data to the canonical data set. In some embodiments, the matrix of data generated by the muscular activity sensing device 208 may simply be multiplied by A to normalize the data to match the orientation of the canonical data set. Hence, data generated by any subsequent configuration/position of the muscular activity sensing device 208 could be normalized by A to be in the same position, and therefore be comparable no matter what orientation in which the muscular activity sensing device 208 was placed on the user's arm. The method 600 then proceeds to an end block and terminates.

FIG. 7 is a block diagram that illustrates aspects of an exemplary computing device 700 appropriate for use with embodiments of the present disclosure. While FIG. 7 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Moreover, those of ordinary skill in the art and others will recognize that the computing device 700 may be any one of any number of currently available or yet to be developed devices.

In its most basic configuration, the computing device 700 includes at least one processor 702 and a system memory 704 connected by a communication bus 706. Depending on the exact configuration and type of device, the system memory 704 may be volatile or nonvolatile memory, such as read only memory (“ROM”), random access memory (“RAM”), EEPROM, flash memory, or similar memory technology. Those of ordinary skill in the art and others will recognize that system memory 704 typically stores data and/or program modules that are immediately accessible to and/or currently being operated on by the processor 702. In this regard, the processor 702 may serve as a computational center of the computing device 700 by supporting the execution of instructions.

As further illustrated in FIG. 7, the computing device 700 may include a network interface 710 comprising one or more components for communicating with other devices over a network. Embodiments of the present disclosure may access basic services that utilize the network interface 710 to perform communications using common network protocols. The network interface 710 may also include a wireless network interface configured to communicate via one or more wireless communication protocols, such as WiFi, 2G, 3G, LTE, WiMAX, Bluetooth, and/or the like.

In the exemplary embodiment depicted in FIG. 7, the computing device 700 also includes a storage medium 708. However, services may be accessed using a computing device that does not include means for persisting data to a local storage medium. Therefore, the storage medium 708 depicted in FIG. 7 is represented with a dashed line to indicate that the storage medium 708 is optional. In any event, the storage medium 708 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 704 and storage medium 708 depicted in FIG. 7 are merely examples of computer-readable media.

Suitable implementations of computing devices that include a processor 702, system memory 704, communication bus 706, storage medium 708, and network interface 710 are known and commercially available. For ease of illustration and because it is not important for an understanding of the claimed subject matter, FIG. 7 does not show some of the typical components of many computing devices. In this regard, the computing device 700 may include input devices, such as a keyboard, keypad, mouse, microphone, touch input device, touch screen, tablet, and/or the like. Such input devices may be coupled to the computing device 700 by wired or wireless connections including RF, infrared, serial, parallel, Bluetooth, USB, or other suitable connections protocols using wireless or physical connections. Similarly, the computing device 700 may also include output devices such as a display, speakers, printer, etc. Since these devices are well known in the art, they are not illustrated or described further herein.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the invention in more detail than is necessary for the fundamental understanding of the system and methods, the description taken with the drawings and/or examples making apparent to those skilled in the art how the several forms of the disclosure may be embodied in practice.

As used herein and unless otherwise indicated, the terms “a” and “an” are taken to mean “one,” “at least one” or “one or more.” Unless otherwise required by context, singular terms used herein shall include pluralities and plural terms shall include the singular.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The embodiments of the invention in which an exclusive property or privilege is claimed are defined as follows:

1. A system for automated administration and evaluation of physical therapy exercises, the system comprising:
a computing device comprising at least one processor and a non-transitory computer readable medium;
a display device communicatively coupled to the computing device;
a hand position sensing device communicatively coupled to the computing device; and
a muscular activity sensing device communicatively coupled to the computing device and configured to sense muscular activity in a forearm of a user.

2. The system of claim 1, wherein the computer-readable medium of the computing device has computer-executable instructions stored thereon that, in response to execution by the at least one processor, cause the computing device to perform actions that include:
presenting, by the computing device via the display device, a motion prompt;
receiving, by the computing device, hand position information from the hand position sensing device;
receiving, by the computing device, muscular activity information from the muscular activity sensing device;
determining, by the computing device, compliance with the motion prompt using the hand position information; and
determining, by the computing device, a muscular activity evaluation score based on the muscular activity information.

3. The system of claim 2, wherein the hand position information includes three-dimensional location information and orientation information for one or more anatomical elements of a hand.

4. The system of claim 2, wherein the muscular activity sensing device includes a plurality of surface electromyography (EMG) sensors.

5. The system of claim 4, wherein the muscular activity information includes a stream of surface EMG sensor values.

6. The system of claim 5, wherein determining a muscular activity evaluation score includes determining a calibration map for the muscular activity information using a modified orthogonal Procrustes technique.

7. The system of claim 2, wherein the actions further include presenting, by the computing device via the display device, an indication of sensed motion based on the received hand position information.

8. The system of claim 2, wherein the actions further include:
setting, by the computing device, a gain level to be used by the hand position sensing device; and
updating, by the computing device, the gain level to be used by the hand position sensing device based on at least one of the determined compliance with the motion prompt and the determined muscular activity evaluation score.

9. A computer-implemented method for automated administration and evaluation of physical therapy exercises, the method comprising:
presenting, by a computing device via a display device, a motion prompt;
receiving, by the computing device, hand position information from a hand position sensing device;
receiving, by the computing device, muscular activity information from a muscular activity sensing device;
determining, by the computing device, compliance with the motion prompt using the hand position information; and
determining, by the computing device, a muscular activity evaluation score based on the muscular activity information.

10. The method of claim 9, wherein the hand position information includes three-dimensional location information and orientation information for one or more anatomical elements of a hand.

11. The method of claim 9, wherein the muscular activity information includes a stream of sensor values from a plurality of surface electromyography (EMG) sensors of the muscular activity sensing device.

12. The method of claim 9, wherein determining a muscular activity evaluation score includes determining a calibration map for the muscular activity information using a modified orthogonal Procrustes technique.

13. The method of claim 9, further comprising presenting, by the computing device via the display device, an indication of sensed motion based on the received hand position information.

14. The method of claim 9, further comprising:
setting, by the computing device, a gain level to be used by the hand position sensing device; and
updating, by the computing device, the gain level to be used by the hand position sensing device based on at least one of the determined compliance with the motion prompt and the determined muscular activity evaluation score.

15. A nontransitory computer-readable medium having computer-executable instructions stored thereon that, in response to execution by one or more processors of a computing device, cause the computing device to perform actions comprising:
presenting, by the computing device via a display device, a motion prompt;
receiving, by the computing device, hand position information from a hand position sensing device;
receiving, by the computing device, muscular activity information from a muscular activity sensing device;
determining, by the computing device, compliance with the motion prompt using the hand position information; and
determining, by the computing device, a muscular activity evaluation score based on the muscular activity information.

16. The computer-readable medium of claim 15, wherein the hand position information includes three-dimensional location information and orientation information for one or more anatomical elements of a hand.

17. The computer-readable medium of claim 15, wherein the muscular activity information includes a stream of sensor values from a plurality of surface electromyography (EMG) sensors of the muscular activity sensing device.

18. The computer-readable medium of claim 17, wherein determining a muscular activity evaluation score includes determining a calibration map for the muscular activity information using a modified orthogonal Procrustes technique.

19. The computer-readable medium of claim 15, wherein the actions further comprise presenting, by the computing device via the display device, an indication of sensed motion based on the received hand position information.

20. The computer-readable medium of claim 15, wherein the actions further comprise:
setting, by the computing device, a gain level to be used by the hand position sensing device; and
updating, by the computing device, the gain level to be used by the hand position sensing device based on at least one of the determined compliance with the motion prompt and the determined muscular activity evaluation score.
